(19) United States
(12) Patent Application Publication
(10) Pub. No.: US 2023/0309841 A1
(43) Pub. Date: Oct. 5, 2023

(54) ENDOTRACHEAL TUBE WITH SENSORS FOR NOCICEPTION STIMULUS FEEDBACK FOR USE IN ANALGESIC DRUG TITRATION

(71) Applicant: Covidien LP, Mansfield, MA (US)

(72) Inventors: Paul S. Addison, Edinburgh (GB); Dean Montgomery, Edinburgh (GB)

(21) Appl. No.: 18/041,428

(22) PCT Filed: Sep. 13, 2021
(86) PCT No.: PCT/US2021/050071

§ 371 Date: Feb. 13, 2023

Related U.S. Application Data
(60) Provisional application No. 63/078,050, filed on Sep. 14, 2020.

(51) Int. Cl. A61B 5/021; A61B 5/024; A61M 16/04

(52) U.S. Cl. CPC A61B 5/021; A61B 5/02405; A61M 16/04; A61B 5/346

(57) ABSTRACT

A patient monitoring system may include processing circuitry configured to receive an indication of a medical event, determine a set of nociception parameters of a patient that corresponds to the medical event, determine a nociception threshold based at least in part on the set of nociception parameters of the patient, compare a nociception parameter of the patient to the nociception threshold to detect a nociception event, and in response to detecting the nociception event, providing an indication to adjust an amount of analgesic administered to the patient.

This application claims priority from U.S. Provisional Patent Application No. 63/078,050, entitled “ENDOTRACHEAL TUBE WITH SENSORS FOR NOCICEPTION STIMULUS FEEDBACK FOR USE IN ANALGESIC DRUG TITRATION” and filed on Sep. 14, 2020, the entire content of which is incorporated herein by reference.

### TECHNICAL FIELD

This disclosure relates to patient monitoring.

### BACKGROUND

Nociception is a response of a sensory nervous system of a subject to certain stimuli, such as chemical, mechanical, or thermal stimuli, that causes the stimulation of sensory nerve cells called nociceptors.

### SUMMARY

The present disclosure describes example devices, systems, and techniques for monitoring the nociception parameters of a patient undergoing a medical procedure using one or more patient-specific nociception threshold values. A clinician may use a nociception monitoring system to monitor the nociception parameters of the patient during a medical procedure to help determine the amount of analgesic to administer to the patient during the surgery. As the patient undergoes the medical procedure, the clinician may administer analgesic to the patient to reduce systemic or other physiological stress experienced by the patient during the medical procedure. While this stress is generally referred to herein as “surgical stress,” the stress may be the result of one or more events occurring during any medical procedure and is not limited to surgery-induced stress responses of a patient. Example nociception parameters include nociception level index (NOL), analgesia nociception index (ANI), surgical pleth index (SPI), composite variability index (CVI), and the like.

The nociception parameters of the patient may correspond to the amount of surgical stress experienced by the patient. When the nociception parameter of the patient increases to be greater than or equal to a nociception threshold, then the nociception parameter may indicate a severe nociceptive stimulus. Correspondingly, the clinician may, in response to the nociception parameter indicating a severe nociceptive stimulus, increase the amount of analgesic being administered to the patient to dampen down the nociception response of the patient and hence reduce the surgical stress caused to the patient.

In some examples, the nociception parameter used for determining whether the patient is experiencing a severe nociceptive stimulus is a predetermined nociception threshold that is determined based on the response of a population of patients to surgical stress. However, different patients may respond differently to surgical stress and stimuli, such that the same level of nociception parameters of different patients may indicate different levels of surgical stress experienced by different patients.

In accordance with aspects of the present disclosure, instead of using a predetermined nociception threshold that is based on a population of patients, to determine whether a patient is experiencing a severe nociceptive stimulus, a clinician may use a nociception threshold that is determined based on the patient's nociceptive response to surgical stress. For example, during a medical procedure, a nociception monitoring system may determine the patient's nociceptive response to intubation and may determine a nociception threshold for the patient based on the patient's nociceptive response. In addition to or instead of the patient's nociceptive response to intubation, a nociception monitoring system may determine the patient's nociceptive response to another medical event, such as an incision or tetanic stimulation (which may be delivered to determine an extent of neuromuscular blockade for anesthesia management). In this way, the nociception monitoring system may determine a nociception threshold that may enable clinician to better determine whether the patient is experiencing a severe nociceptive stimulus compared with the use of a predefined threshold.

In some aspects, a method comprises: monitoring, by processing circuitry, nociception parameters of a patient during a medical procedure; receiving, by the processing circuitry, an indication of a medical event; determining, by the processing circuitry, a set of nociception parameters of the patient that corresponds to the medical event; determining, by the processing circuitry, a nociception threshold based at least in part on the set of nociception parameters of the patient; comparing, by the processing circuitry, a nociception parameter of the patient to the nociception threshold to detect a nociception event; and in response to detecting the nociception event, providing, by the processing circuitry, an indication to adjust an amount of analgesic administered to the patient.

In some examples, a system comprises: memory; and processing circuitry operably coupled to the memory and configured to: monitor nociception parameters of a patient during a medical procedure; receive an indication of a medical event; determine a set of nociception parameters of the patient that corresponds to the medical event; determine a nociception threshold based at least in part on the set of nociception parameters of the patient; compare a nociception parameter of the patient to the nociception threshold to detect a nociception event; and in response to detecting the nociception event, provide an indication to adjust an amount of analgesic administered to the patient.

In some aspects, a non-transitory computer readable storage medium comprises instructions that, when executed, cause processing circuitry to: monitor nociception parameters of a patient during a medical procedure; receive an indication of a medical event; determine a set of nociception parameters of the patient that corresponds to the medical event; determine a nociception threshold based at least in part on the set of nociception parameters of the patient; compare a nociception parameter of the patient to the nociception threshold to detect a nociception event; and in response to detecting the nociception event, provide an indication to adjust an amount of analgesic administered to the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a block diagram illustrating an example environment in which a patient monitoring system monitors one or more nociception parameters of a patient undergoing a medical procedure.

FIGS. 2A-2C illustrate an example technique for determining a nociception threshold for a patient, in accordance with aspects of this disclosure.

FIG. 3 is a block diagram illustrating the patient monitoring system of FIG. 1.

FIG. 4 is a flow diagram illustrating an example method of determining whether to increase the amount of analgesic administered to patient undergoing a medical procedure.

### DETAILED DESCRIPTION

Aspects of the present disclosure describe techniques for monitoring the nociception parameters of a patient undergoing a medical procedure, such as surgery, to help determine the amount of analgesic to administer to the patient during the medical procedure. Nociception monitors may provide a continuous measure of a nociception parameter for a patient undergoing a medical procedure in order to track the nociception response of the patient. The nociception parameter can be based on one or more physiological signals, such as an electrocardiogram (ECG), a photoplethysmogram (PPG), electroencephalogram (EEG), skin conductance, body temperature, and the like, and may typically be displayed over time. A clinician may monitor the nociception parameter of a patient to determine the amount of analgesic to administer to the patient during the medical procedure. As the patient undergoes the medical procedure, the clinician may administer analgesic to the patient to reduce stress experienced by the patient during the medical procedure. While this stress is generally referred to herein as “surgical stress,” the stress may be the result of any medical procedure and is not limited to surgery-induced stress responses of a patient. The stress can be, for example, an activation of a patient's sympathetic nervous system, an endocrine response, and/or immunological or hematological change in the patient.

A clinician may use a nociception monitoring system to monitor the nociception parameter of the patient during the medical procedure, and the clinician may determine whether to adjust the amount of analgesic to administer to the patient based on the nociception parameter of the patient. In some examples, the clinician may monitor the nociception parameter of the patient to determine whether the nociception parameter of the patient increases above a nociception threshold, which may indicate a severe nociceptive stimulus experienced by the patient. The clinician may, in response to the nociception parameter of the patient increasing above the nociception threshold, adjust (e.g., increase) the amount of analgesic to dampen the nociception stimulus experienced by the patient.

Noise in the nociception parameters may occasionally cause false positive indications of a severe nociceptive stimulus. Such noise may be caused by patient motion, electrocautery, administration of drugs to the patient, and the like, or may be present in underlying signals from which the nociception parameters are derived. For example, such noise may cause the nociception monitoring system to sense increases in the nociception parameters of the patient above the nociception threshold even when there is not a corresponding increase in the surgical stress experienced by the patient. If the clinician were to increase the amount of analgesic administered to the patient in response to such false positive indications of a severe nociceptive stimulus, the clinician may unwittingly administer additional analgesic to the patient where it may not be required.

In addition, different patients may respond differently to surgical stress and stimuli, such that the same level of nociception parameters of different patients may indicate different levels of surgical stress experienced by different patients. These different responses may be due to the physiology of patients, the amount of analgesic already administered to the patients, and the like. As such, using the same nociception threshold, such as a nociception threshold determined based on the response of a population of patients to surgical stress, to determine whether different patients are experiencing a severe nociceptive stimulus may lead clinicians to administer additional analgesic to patients where it may not be required or may lead clinicians to less timely administer additional analgesic to patient experiencing severe nociceptive stimuli.

This disclosure describes devices, systems, and methods for adaptively determining patient-specific nociception thresholds for patients in ways that enable clinicians to more accurately adjust the amount of analgesic administered to the patients. Specifically, aspects of this disclosure describe techniques that adaptively determines the nociception threshold for a patient based at least in part on the patient's nociception parameter in response to a medical event, such as intubation, extubation, the patient being incised, or tetanic stimulation delivered to monitor neuromuscular blockade of the patient. The medical event may be associated with an increase in a stress response of the patient, such that a nociception threshold for detecting a relatively severe nociception stimulus experienced by the patient can be determined based on one or more nociception parameters (referred to herein as a “set” of nociception parameters) corresponding to the medical event (e.g., corresponding in time).

As part of the medical procedure, a patient may be intubated using an endotracheal tube to maintain an open airway to assist the patient with breathing or to serve as a conduit through which a clinician may administer drugs or medication. At the end of the medical procedure, the patient may be extubated to remove the endotracheal tube from the patient. Intubation and extubation may be relatively major noxious stimuli that stimulate nociception in the patient and cause the nociception parameter of the patient to increase during intubation and extubation. As such, a nociception monitoring system may monitor the nociception parameter of the patient during intubation and/or extubation and determine, based on the nociception parameter of the patient, a nociception threshold that is adapted for the patient.

The nociception parameter of the patient may also increase during other medical events, such as an incision event (when an incision is made in the patient), a tetanic stimulation event when an electrical stimulation device delivers electrical stimulation to the patient in order to monitor a level of neuromuscular blockade resulting from the delivery of anesthesia to the patient, any stimulation which triggers nociception (e.g., pressing on an organ, excising tissue, cauterizing, etc.) or any combination of medical events described here. A nociception monitoring system may monitor the nociception parameter of the patient during the medical event and, based on the corresponding set of nociception parameters of the patient, determine a patient-specific nociception threshold that can be subsequently used to monitor the nociception parameter of the patient and detect a severe nociception response of the patient.

By determining a nociception parameter that is adapted for the patient based at least in part on the nociception parameter of the patient during a medical event, the devices, systems, and techniques of this disclosure may increase the accuracy of the nociception monitoring system in associating nociception parameters of a patient with real increases in the surgical stress experienced by the patient. Increasing the accuracy of the nociception monitoring system may lead to positive outcomes for the patient by at least enabling a clinician or an analgesic administration system to more accurately and timely administer analgesic to the patient when it may be required to reduce the surgical stress caused to the patient and to decrease unnecessary administration of additional analgesic administered to the patient due to false positives.

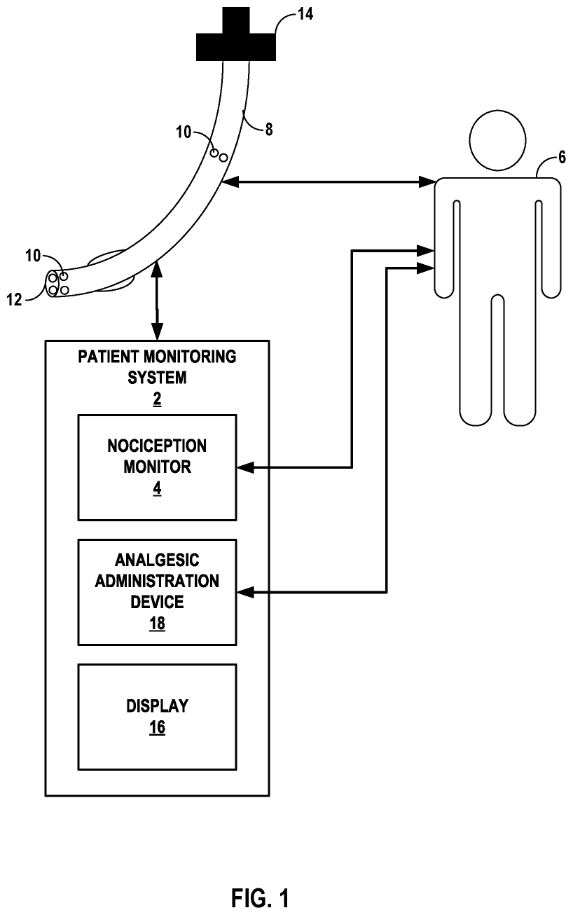

FIG. 1 is a block diagram illustrating an example environment in which a patient monitoring system monitors one or more nociception parameters of a patient undergoing a medical procedure. As shown in FIG. 1, patient monitoring system 2 may monitor one or more physiological signals of patient 6 to determine the amount of surgical stress caused by the surgery to patient 6. By monitoring the amount of surgical stress experienced by patient 6, patient monitoring system 2 or a clinician that uses patient monitoring system 2 may be able to determine whether to increase the amount of analgesic to administer to the patient during the surgery.

Patient monitoring system 2 is configured to monitor patient 6 during surgery and configured to titrate analgesic or anesthetic delivered to patient 6 during surgery to provide anesthesia for patient 6. Patient monitoring system 2 may include nociception monitor 4, analgesic administration device 18, and display 16. As the clinician performs a medical procedure on patient 6, nociception monitor 4 of patient monitoring system 2 may monitor the amount of surgical stress experienced by patient 6 by monitoring one or more physiological signals of patient 6, such as, but not limited to one or more of an ECG, a PPG, an EEG, the skin conductance of patient 6, the body temperature of patient 6, a respiratory rate, and the like, to determine a continuous measure of a nociception parameter associated with patient 6 during the surgery, where the nociception parameter corresponds to the amount of surgical stress experienced by patient 6. In some examples, the nociception parameter may be an integer, and may range from, for example, 0 to 100. As such, by determining a continuous measure of a nociception parameter associated with patient 6 during the surgery, nociception monitor 4 may determine a continuous measure of the amount of surgical stress experienced by patient 6 during surgery.

Display 16 is configured to display the nociception parameter over time. For example, as nociception monitor 4 determines the nociception parameter associated with patient 6, display 16 may output a graphical representation of the nociception parameter over time, which may be viewed by a clinician to monitor the amount of surgical stress experienced by patient 6.

In some examples, patient monitoring system 2 may include analgesic administration device 18, which may include one or more components and/or devices for administering analgesic to patient 6 during surgery. Analgesic administration device 18 may be coupled to patient 6, such as via one or more intravenous (IV) lines, a breathing mask, a tube, and the like, to titrate analgesic to patient 6 in order to provide anesthesia to patient 6 during surgery.

In some examples, the analgesic administration device 18 may be able to administer analgesic to patient 6 without user intervention from, for example, a clinician. That is, patient monitoring system 2 may control the amount of analgesic being administered by analgesic administration device 18 to patient 6 (i.e., automatically titrate analgesic delivered to patient 6), such as increasing the amount of analgesic administered by analgesic administration device 18 to patient 6 or decreasing the amount of analgesic administered by analgesic administration device 18 to patient 6, without user intervention.

In some examples, a clinician may control the amount of analgesic being administered by analgesic administration device 18 to patient 6. For example, the clinician may provide user input to patient monitoring system 2 indicative of the amount of analgesic being administered by analgesic administration device 18 to patient 6. Patient monitoring system 2 may receive such user input indicative of the amount of analgesic being administered by analgesic administration device 18 to patient 6 and may, in response, control analgesic administration device 18 to administer the amount of analgesic to patient 6 indicated by the user input.

As a medical procedure is performed on patient 6, nociception monitor 4 of patient monitoring system 2 may continuously determine the nociception parameter associated with patient 6 in order to monitor the amount of surgical stress experienced by patient 6. Nociception monitor 4 may specify a nociception threshold for patient 6, where nociception parameters of patient 6 that are at or above the nociception threshold may be indicative of patient 6 experiencing a severe nociceptive stimulus. In the example where the nociception parameter of patient 6 may range from 0 to 100, a nociception threshold may also be an integer value between 0 and 100, such as 70, 80, and the like. As such, if nociception monitor 4 determines that the nociception parameter of patient 6 is greater than or equal to the nociception threshold, then patient monitoring system 2 may detect that a nociception event has occurred and may accordingly cause analgesic administration device 18 to increase the amount of analgesic administered to patient 6 to dampen down the surgical stress experienced by patient 6 and to decrease the nociception parameter of patient 6 to below the nociception threshold. In other examples, if nociception monitor 4 determines that the nociception parameter of patient 6 is greater than or equal to the nociception threshold, then patient monitoring system 2 may provide an indication via display 16 or another user output device (e.g., audio circuitry configured to generate an audible output or circuitry configured to generate a tactile output perceived by a clinician) to indicate to adjust an amount of analgesic administered to the patient.

However, different patients may respond differently to surgical stress and stimuli, such that the same level of nociception parameters of different patients may indicate different levels of surgical stress experienced by different patients. These different responses may be due to the physiology of patients, the amount of analgesic already administered to the patients, and the like. Thus, using the same nociception threshold to determine whether different patients are experiencing a severe nociceptive stimulus may lead to suboptimal results.

In accordance with aspects of this disclosure, instead of using a preset, non-patient specific nociception threshold to monitor a nociception parameter of patient 6, patient monitoring system 2 is configured to adaptively determine a nociception threshold for patient 6 based at least in part on patient 6's response to surgical stress and stimuli. Patient monitoring system 2 may monitor the nociception parameter of patient 6 during a medical event and determine a nociception threshold for patient 6 based at least in part on the surgical stress experienced by patient 6 during the medical event. The medical event can be, for example, intubation or extubation such that the surgical stress is caused by the intubation or the extubation, respectively. As another example, in addition or instead of intubation or extubation, the medical event can be an incision event (when a physiological feature of the patient, such as skin, is cut) or an electrical stimulation event (e.g., when tetanic stimulation delivered to monitor neuromuscular blockade of the patient).

As part of a medical procedure, a clinician may intubate patient 6 by inserting endotracheal tube 8 through patient 6's mouth and into patient 6's trachea to assist patient 6 with breathing or to serve a conduit through which a clinician may administer drugs or medication to patient 6. Endotracheal tube 8 may include fitting 14 at a proximal end of endotracheal tube 8 configured to be connected to a pressurized gas source, such as a pressurized oxygen source, and such pressurized gas or oxygen may flow through a lumen defined by endotracheal tube 8 and out of opening 12 at a distal end of endotracheal tube 8 to output the pressurized gas or oxygen to patient 6's trachea.

In some examples, one or more sensors 10, such as accelerometers, force sensors, conductive sensors, pressure sensors, and the like may be mounted or otherwise coupled to endotracheal tube 8 to sense intubation and/or extubation of endotracheal tube 8. One or more sensors 10 may measure linear acceleration of endotracheal tube 8, the amount of force exerted by endotracheal tube 8, and the like, during intubation and/or extubation of patient 6, and may generate one or more signals indicative of linear acceleration of endotracheal tube 8, the amount of force exerted by endotracheal tube, and the like that circuitry of endotracheal tube 8 may be configured to send to patient monitoring system 2. Thus, in examples, one or more sensors 10 do not sense a physiological signal of patient 6, but, rather, sense forces applied to endotracheal tube 8, movement of endotracheal tube 8, or the like to give context to sensed nociception parameters. The context can indicate, for example, that a particular set of nociception parameters (one or more nociception parameters) corresponds to a medical event that caused an increase in surgical stress to patient 6. The correspondence can be, for example, in time or in cause/effect relationship that may not necessarily overlap in time.

One or more sensors 10 may be positioned at any suitable position along endotracheal tube 6. In some examples, one or more sensors 10 may be mounted at or near the distal end of endotracheal tube 8 near opening 12. In other examples, one or more sensors 10 may be mounted at or near the proximal end of tracheal tube 8 near fitting 14, or at or near any other suitable location between the proximal and distal ends of tracheal tube 8. In some examples, one or more sensors 10 may be mounted both at or near the distal end of endotracheal tube 8 near opening 12 and at or near the proximal end of tracheal tube 8 near fitting 14, or any other suitable location on endotracheal tube 8, such as locations to facilitate one or more sensors 10 generating signals that indicate endotracheal tube 8 has been inserted in patient 6.

Patient monitoring system 2 may be operably coupled to one or more sensors 10 of endotracheal tube 8 via a wired or wireless network or via any other communications medium to communicate with patient monitoring system 2. In some examples, circuitry of endotracheal tube 8 is configured to continuously send indications of sensor values, which may correspond to the linear acceleration of endotracheal tube 8, the amount of force exerted by endotracheal tube 8, and the like, measured by one or more sensors 10 to patient monitoring system 2.

Patient monitoring system 2 may be configured to receive indications of sensor values generated by one or more sensors 10 and to detect, based at least in part on the sensor values, an intubation event that corresponds to patient 6 being intubated with endotracheal tube 8 and/or an extubation event that corresponds to endotracheal tube 8 being removed from patient 6. For example, if the sensor values include linear acceleration values generated by one or more accelerometers, patient monitoring system 2 may be configured to determine whether the linear acceleration values received from endotracheal tube 8 are indicative of patient 6 being intubated and/or extubated, such as by determining that a sharp increase in the linear acceleration values received from endotracheal tube 8 is indicative of patient 6 being intubated with endotracheal tube 8, and a subsequent sharp increase in the linear acceleration values received from endotracheal tube is indicative of endotracheal tube 8 being extubated from patient 6. The sharp increase in sensor values, can be, for example, a rate of change in the sensor values being greater than or equal to a rate of change threshold stored by patient monitoring system 2 or another device in communication with patient monitoring system 2.

In another example, if the sensor values include force values generated by one or more force sensors, then patient monitoring system 2 may be configured to determine whether the force values are indicative of patient 6 being intubated and/or extubated. For example, patient monitoring system 2 may determine that a sharp increase in the force values received from endotracheal tube 8 is indicative of patient 6 being intubated with endotracheal tube 8, and a subsequent sharp increase in the force values received from endotracheal tube is indicative of endotracheal tube 8 being extubated from patient 6.

Patient monitoring system 2 may therefore be able to determine an intubation period for patient 6, which is the period of time within which the intubation event takes place. During the intubation period, the placement of endotracheal tube 8 in patient's 6 trachea may cause the amount of physiological stress experienced by patient 6 to increase, and may correspondingly cause the level of the nociception parameter of patient 6 to also increase. By determining the intubation period, patient monitoring system 2 may be configured to determine the set of nociception parameters of patient 6 that corresponds to the intubation event.

Similarly, patient monitoring system 2 may be able to determine an extubation period for patient 6, which is the period of time within which the intubation event takes place, based on the signals generated by one or more sensors 10. Patient monitoring system 2 may then determine the set of nociception parameters of patient 6 that corresponds to the extubation event based on the set of nociception parameters of patient 6 generated by nociception monitor 4 during the extubation period.

In other examples, the medical event can include an incision event or electrical stimulation event, as discussed above. In these examples, processing circuitry of patient monitoring system 2 may detect the medical event based on input from another device, such as a surgical robot or an electrical stimulation device, based on user input, or the like. In response, patient monitoring system 2 may determine the set of nociception parameters of patient 6 that corresponds to the medical event based on the set of nociception parameters of patient 6 generated by nociception monitor 4 during a time period determined based on the input and indicative of patient's 6 stress response to the medical event. For example, the time period can begin at the time system 2 receives the input and extend over a predetermined period of time (e.g., 5 seconds to 60 seconds). As another example, the time period can begin prior to the user input (e.g., system 2 or monitor 4 can include a memory that stores historical nociception parameters), such as a window of about 1 second or 60 seconds prior to the input. Other time periods can be used in other examples.

Processing circuitry of patient monitoring system 2 may be configured to determine, based at least in part on a set of nociception parameters of patient 6 that corresponds to the intubation event or other medical event, a nociception threshold for the patient. For example, processing circuitry of patient monitoring system 2 may be configured to derive a characteristic nociception parameter (NPS) for patient 6 based at least in part on the set of nociception parameters of patient 6 during the intubation period (or other time period corresponding to a medical event), and may be configured to determine a nociception threshold for patient 6 based at least in part on the characteristic nociception parameter for patient 6.

In some examples, processing circuitry of patient monitoring system 2 may be configured to determine a characteristic nociception parameter for patient 6 as a multiple of the mathematical mean of the set nociception parameters that corresponds to the medical event, also referred to as a mean nociception parameter, such as 0.5 times the mean of the set of nociception parameters that corresponds to the medical event, 1.0 times the mean of the set of nociception parameters that corresponds to the medical event, 1.5 times the mean of the set of nociception parameters that corresponds to the medical event, or 2.0 times the mean of the set of nociception parameters that corresponds to the medical event.

In some examples, processing circuitry of patient monitoring system 2 may be configured to determine a characteristic nociception parameter for patient 6 as a percentile of the set of nociception parameters that corresponds to the medical event, such as the 50^{th}, 75^{th}, 90^{th}, or 95^{th} percentile of the set of nociception parameters that corresponds to the medical event. Processing circuitry of patient monitoring system 2 may also be configured to use any other suitable distance metrics and multiples thereof to derive a characteristic nociception parameter for patient 6 based at least in part on the set of nociception parameters of patient 6 that corresponds to the medical event.

In some examples, to determine a nociception threshold for patient 6 based at least in part on the characteristic nociception parameter for patient 6, processing circuitry of patient monitoring system 2 may be configured to determine the nociception threshold for patient 6 as the characteristic nociception parameter for patient 6. That is, processing circuitry of patient monitoring system 2 may be configured to set the value of the nociception threshold for patient 6 to the value of the characteristic nociception parameter for patient 6. In other examples, processing circuitry of patient monitoring system 2 may be configured to determine the nociception threshold for patient 6 as a percentage of the characteristic nociception parameter for patient 6, such as 90% of the characteristic nociception parameter, 105% of the characteristic nociception parameter, and the like, or as a multiple of the characteristic nociception parameter for patient 6, such as 0.95 times the characteristic nociception parameter, 1.08 times the characteristic nociception parameter, and the like.

In some examples, processing circuitry of patient monitoring system 2 is also be configured to adjust the nociception threshold based on various factors. In some examples, if one or more sensors 10 coupled to endotracheal tube 8 comprises force sensors that measure the amount of force exerted on patient 6 by intubating and/or extubating endotracheal tube 8, then processing circuitry of patient monitoring system 2 may be configured to adjust the nociception threshold based on the amount of force exerted on patient 6 by intubating and/or extubating endotracheal tube 8 as measured by the force sensors. That is, the force sensed during the intubation or extubation may provide context for the relative amount of surgical stress experienced by patient 6 during the particular intubation or extubation event.

Processing circuitry of patient monitoring system 2 may be configured to determine whether the amount of force exerted on patient 6 by intubating and/or extubating endotracheal tube 8 as measured by the force sensors is relatively high or relatively low, e.g., using a predetermined force threshold. A relatively low amount of force measured by the force sensors (less than or equal to the force threshold) may indicate that relatively less force was used to intubate patient 6 with endotracheal tube 8, which may cause patient 6 to exhibit a relatively lower nociception response due to intubating endotracheal tube 8. Thus, if processing circuitry of patient monitoring system 2 determines that the force sensors measured a relatively low amount of force, such that the amount of force is less than or equal to a predetermined force threshold during intubation of patient 6 with endotracheal tube 8, then processing circuitry of patient monitoring system 2 may be configured to adjust the nociception threshold by raising the nociception threshold to account for the relatively lower nociception response of patient 6 due to intubating endotracheal tube 8.

In contrast, a relatively high amount of force measured by the force sensors may indicate that relatively more force was used to intubate patient 6 with endotracheal tube 8, which may cause patient 6 to exhibit a relatively higher nociception response due to intubating endotracheal tube 8. Thus, if processing circuitry of patient monitoring system 2 determines that the force sensors measured a relatively high amount of force, such that the amount of force is greater than the predetermined force threshold, during intubation of patient 6 with endotracheal tube 8, then processing circuitry of patient monitoring system 2 may be configured to adjust the nociception threshold by lowering the nociception threshold to account for the relatively higher nociception response of patient 6 due to intubating endotracheal tube 8.

In some examples, processing circuitry of patient monitoring system 2 may be configured to adjust the nociception threshold based on the amount of analgesic already administered to patient 6 prior to the intubation period for patient 6. For example, a patient having a relatively higher analgesic load relative to, for example, an analgesic load threshold, may not exhibit as large of a nociception response to intubation or other medical event as a patient having a relatively lower analgesic load. Such an analgesic load threshold may vary based on the patient, the analgesic used, the surgery type, and the like. As such, if processing circuitry of patient monitoring system 2 determines that the amount of analgesic administered to patient 6 prior to the intubation period or other medical event period is relatively high, such as being above a high analgesic load threshold, then the processing circuitry of patient monitoring system 2 may be configured to adjust the nociception threshold by raising the nociception threshold. Conversely, if processing circuitry of patient monitoring system 2 determines that the amount of analgesic administered to patient 6 prior to the intubation period or other medical event period is relatively low, such as being below a low analgesic load threshold, then the processing circuitry of patient monitoring system 2 may be configured to adjust the nociception threshold by lowering the nociception threshold.

In some examples, processing circuitry of patient monitoring system 2 is configured to adjust, based at least on the analgesic agent and/or the site on patient 6 where the analgesic is administered, the nociception parameter over time. In some examples, patient monitoring system 2 may be configured to receive input indicative of the analgesic agent administered to patient 6 and/or the site on patient 6 where the analgesic is administered. In some examples, patient monitoring system 2 may communicate with an electronic medical records system to receive information indicative of the analgesic agent administered to patient 6 and/or the site on patient 6 where the analgesic is administered. In some examples, patient monitoring system 2 may store a lookup table that contains information regarding analgesia, administration sites, and how long it takes for analgesia to wear off given the different types of analgesia and different administration sites.

In some examples, processing circuitry of patient monitoring system 2 may be configured to determine the characteristic nociception parameter for patient 6 at multiple points during a medical procedure and to determine the nociception threshold for patient 6 based at least in part on the determined characteristic nociception parameter at those points during the medical procedure. For example, at a point in time during the medical procedure, processing circuitry of patient monitoring system 2 may be configured to determine the nociception parameter during a period of time that immediately precedes the point in time, and may be configured to determine the characteristic nociception parameter based on the nociception parameter during that period of time. Processing circuitry of patient monitoring system 2 may therefore be configured to determine the nociception threshold for patient 6 based at least in part on the determined characteristic nociception parameter, as discussed above.

In some examples, patient monitoring system 2 may be able detect the occurrence of an intubation event without receiving an indication of sensor values from endotracheal tube 8. In some examples, endotracheal tube 8 and/or patient monitoring system 2 may be operably coupled to an input device, such as a physical switch, a physical control, a tablet computer, etc. that a clinician may provide user input, such as by flipping the switch, providing user input at the tablet computer, and the like at the time of intubation to indicate the occurrence of the intubation event. Patient monitoring system 2 may receive an indication of such a user input, such as from endotracheal tube 8 or from the input device to determine the occurrence of an intubation event.

While aspects of this disclosure are described with respect to an intubation event, the techniques described in this disclosure are equally applicable to determining the nociception parameter for patient 6 based on monitoring the nociception response of patient 6 to any other medical event, such as incisions made in patient 6, applied tetanic stimulation, and the like, including the intubation event. When a medical event occurs, patient monitoring system 2 may determine that the medical event has occurred in any suitable fashion.

In the example of an incision made in patient 6, the surgical instrument making the incision may include one or more force sensors configured to generate a signal indicative of the force applied by the surgical instrument to patient 6. Patient monitoring system 2 may receive indications of such force values sensed by the one or more sensors and determine that a medical event has occurred based on the force values. In another example, a clinician may provide user input, such as by flipping a switch, providing user input at a tablet computer, and the like at the time of the medical event to indicate the occurrence of the medical event, and patient monitoring system 2 may receive an indication of such a user input to determine the occurrence of a medical event. Patient monitoring system 2 may therefore determine the set of nociception parameters of patient 6 that corresponds to the medical event, determine a characteristic nociception parameter based on the set of nociception parameters of patient 6, and determine the nociception threshold for patient 6 based at least in part on the characteristic nociception parameter of patient 6, as described throughout this disclosure.

As described above, processing circuitry of patient monitoring system 2 may be configured to determine, based at least in part on comparing the nociception parameter of patient 6 to the nociception threshold, whether a nociception event has occurred. Processing circuitry of patient monitoring system 2 may be configured to obtain a nociception parameter of patient 6, such as by receiving the nociception parameter of patient 6 from nociception monitor 4. The processing circuitry of patient monitoring system 2 may detect a nociception event has occurred by at least comparing the nociception parameter of patient 6 to the nociception threshold for patient 6, such as by determining that a nociception parameter of patient 6 is greater than or equal to the nociception threshold.

Patient monitoring system 2 may, in response to determining an occurrence of a nociception event for patient 6, provide an indication of the nociception event, such as by generating and presenting an alert via display 16 or another output device including output circuitry. In some examples, patient monitoring system 2 may, in response to determining an occurrence of a nociception event for patient 6, provide an indication to adjust an amount of analgesic to administer to patient 6 via display or another output device including output circuitry. Thus, in these examples, if patient monitoring system 2 determines that the nociception parameter of patient 6 is greater than or equal to the nociception threshold, then patient monitoring system 2 may provide an indication to adjust an amount of analgesic to administer to patient 6, such as by providing an indication to increase the amount of analgesic to administer to patient 6.

In some examples, a clinician may manually control analgesic administration device 18 to administer analgesic to patient 6. As such, in order to provide an indication to adjust an amount of analgesic to administer to patient 6, patient monitoring system 2 may output, for display at display 16, an indication to a clinician to adjust the amount of analgesic administered to patient 6. For example, patient monitoring system 2 may output, for display at display 16, an indication of the amount of analgesic to administer to patient 6 or a more general instruction or suggestion to the clinician to increase or otherwise adjust the amount of analgesic.

In some examples, patient monitoring system 2 may be able to control analgesic administration device 18 to administer analgesic to patient 6 without user intervention. As such, in order to provide an indication to adjust an amount of analgesic to administer to patient 6, patient monitoring system 2 may output a signal to analgesic administration device 18 to direct analgesic administration device 18 to increase or otherwise adjust the amount of analgesic to administer to patient 6. Analgesic administration device 18 may, in response to receiving the signal, increase or otherwise adjust the amount of analgesic to administer to patient 6.

In some examples, patient monitoring system 2 may determine how much to adjust the amount of analgesic administered to patient 6 and/or whether to adjust the amount of analgesic administered to patient 6 based on the current level of analgesic administered to patient 6 and/or the total amount of analgesic administered to patient 6 during the current medical procedure. In some examples, patient monitoring system 2 may limit the amount of analgesic administered to patient 6 at any point in time to a specified analgesic level. As such, patient monitoring system 2 may increase the amount of analgesic administered to patient 6 at a point in time to no more than the specified analgesic level. If patient monitoring system 2 determines that increasing the amount of analgesic administered to patient 6 would cause the amount of analgesic administered to patient 6 to rise above the specified analgesic level, then patient monitoring system 2 may refrain from increasing the amount of analgesic administered to patient 6 or providing an instruction to increase the amount of analgesic via display 16.

In some examples, patient monitoring system 2 may determine how much to adjust the amount of analgesic administered to patient 6 and/or whether to adjust the amount of analgesic administered to patient 6 based on the total amount of analgesic administered to patient 6 during the course of the surgery or other medical procedure. For example, the total amount of analgesic administered to patient 6 over the course of the surgery may not exceed a total analgesic limit. If patient monitoring system 2 determines that increasing the amount of analgesic administered to patient 6 would cause the total amount of analgesic administered to patient 6 over the course of the surgery to rise above the total analgesic limit, then patient monitoring system 2 may refrain from increasing the amount of analgesic administered to patient 6 providing an instruction to increase the amount of analgesic via display 16.

Patient monitoring system 2 may determine how much to increase the amount of analgesic administered to patient 6 using any techniques described above alone or in combination with each other. Further, while the techniques described above are described with respect to intubation of patient 6, the techniques described above are equally applicable to other noxious stimuli that may occur during a medical procedure, such as incisions and applied tetanic stimulation.

The techniques described herein may provide one or more advantages. By communicating with endotracheal tube 8, patient monitoring system 2 may be able to determine when an intubation event occurs and may adaptively determine a patient-specific nociception threshold for patient 6 based on the nociception response of patient 6 during the intubation event. Similarly, in the case of other types of medical events, such as extubation events, incision events, or electrical stimulation events, patient monitoring system 2 may adaptively determine a patient-specific nociception threshold for patient 6 based on the nociception response of patient 6 during the particular medical event.

Determining a patient-specific nociception threshold for patient 6 may enable patient monitoring system 2 to more effectively dampen excess surgical stress experienced by patient 6 compared with using a predetermined nociception threshold that is not specific to patient 6, and may enable patient monitoring system 2 to better administer (e.g., more timely) the proper amount of analgesic to patient 6. The proper amount of analgesic can be, for example, an amount of analgesic necessary to provide the desired analgesia outcomes for patient 6 but not being too much analgesic, which may lead to undesirable outcomes for patient 6.

Administering a more proper amount of analgesic to the patient (e.g., better corresponding to surgical stress experienced by patient 6 during surgery using the techniques described herein may have one or more beneficial outcomes, such as leading to reductions in opioid administration during and after surgery, post-operative pain scores, the length of the hospital stay, and/or post-operative complications.

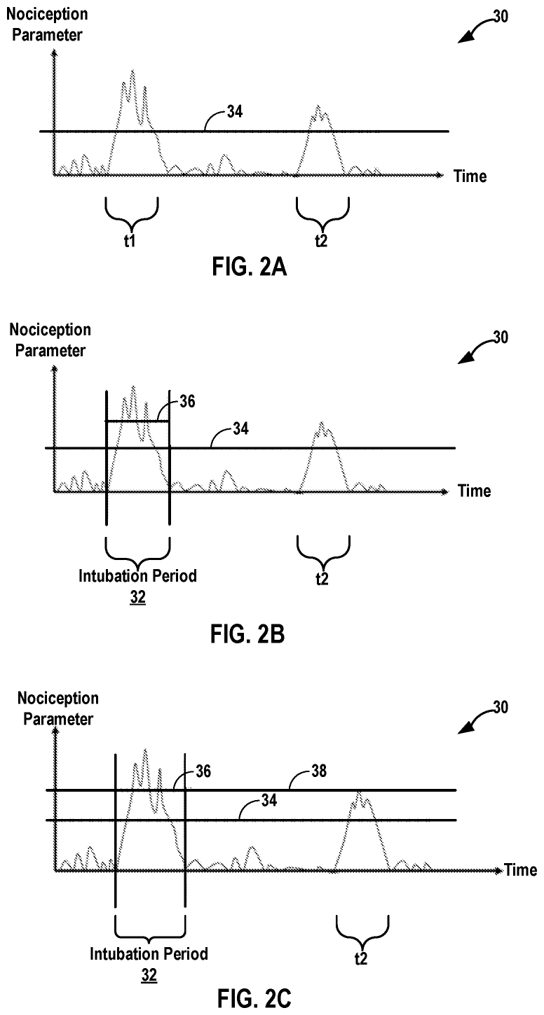

FIGS. 2A-2C illustrate an example technique for determining a patient-specific nociception threshold for a patient, in accordance with aspects of this disclosure. As shown in FIG. 2A, time graph 30 is a visual representation of the nociception parameter of patient 6 over time during a medical procedure, such as monitored by nociception monitor 4. In the example of FIG. 2A, the nociception parameter may be greater than or equal to preset nociception threshold 34 during time period t1 and during time period t2. As such, if patient monitoring system 2 uses preset nociception threshold 34 for detecting nociception events, then patient monitoring system 2 may detect nociception events for patient 6 during time period t1 and time period t2.

As shown in FIG. 2B, patient monitoring system 2 may determine that time period t1 is an intubation period 32. Accordingly, patient monitoring system 2 may determine characteristic nociception parameter 36 based at least in part on the nociception parameter that corresponds to intubation period 32. For example, patient monitoring system 2 may determine characteristic nociception parameter 36 based at least in part on the nociception parameter during intubation period 32 (and, in some examples, not outside of intubation period 32). In the example of FIG. 2B, characteristic nociception parameter 36 may be greater than the preset nociception threshold 34. In other examples, characteristic nociception parameter 36 may be less than the preset nociception threshold 34.

As shown in FIG. 2C, patient monitoring system 2 may determine a nociception threshold 38 for patient 6 and specific to actual nociception parameters of patient 6 based on the characteristic nociception parameter 36. In the example of FIG. 2C, patient monitoring system 2 may set the value of nociception threshold 38 to the value of characteristic nociception parameter 36. If patient monitoring system 2 uses nociception threshold 38 for detecting the occurrence of nociception events instead of using nociception threshold 34, then patient monitoring system 2 may not detect a nociception event during time period t2 because the nociception parameter during time period t2 remains below nociception threshold 38. In other examples, patient monitoring system 2 may set the value of nociception threshold 38 to the based on the value of characteristic nociception parameter 36, but not equal to characteristic nociception parameter 36. As discussed above, for example, characteristic nociception parameter 36 can be a percentage of characteristic nociception parameter 36 or a multiple of characteristic nociception parameter 36.

As discussed above, patient monitoring system 2 may determine a nociception threshold 38 for patient 6 that is different from the nociception threshold 34 based on the nociception parameter that corresponds to intubation period 32. For example, the amplitude of the nociception parameter that corresponds to intubation period 32 may be affected by the amount of force exerted on patient 6 by intubating endotracheal tube 8. A relatively low amount of force used to intubate patient 6 may cause patient 6 to exhibit a relatively lower nociception response during the intubation period 32, and patient monitoring system 2 may, in response, raise the nociception threshold 38 to account for the relatively lower nociception response of patient 6 that corresponds to intubation period 32. In contrast, a relatively high amount of force used to intubate patient 6 may cause patient 6 to exhibit a relatively higher nociception response during the intubation period 32, and patient monitoring system 2 may, in response, lower the nociception threshold 38 to account for the relatively higher nociception response of patient 6 that corresponds to intubation period 32.

Similarly, the amount of analgesic already administered to patient 6 prior to the intubation period 32 may affect the amplitude of the nociception parameter that corresponds to intubation period 32 and the resulting nociception threshold 38. For example, a patient having a relatively higher analgesic load relative to, for example, an analgesic load threshold, may not exhibit as large of a nociception response that corresponds to intubation period 32 as a patient having a relatively lower analgesic load. As such, if that the amount of analgesic administered to patient 6 prior to the intubation period 32 is relatively high, the nociception response that corresponds to intubation period 32 may have a relatively lower amplitude, and patient monitoring system 2 may, in response, raise the nociception threshold 38 to account for the relatively lower nociception response of patient 6 that corresponds to intubation period 32. Conversely, if that the amount of analgesic administered to patient 6 prior to the intubation period 32 is relatively low, the nociception response that corresponds to intubation period 32 may have a relatively higher amplitude, and patient monitoring system 2 may, in response, lower the nociception threshold 38 to account for the relatively higher nociception response of patient 6 that corresponds to intubation period 32.

In some examples, processing circuitry of patient monitoring system 2 may be configured to adjust the nociception threshold over time. For example, as the analgesic previously administered to patient 6 wears off over time, processing circuitry of patient monitoring system 2 adjust the nociception threshold over time. In some examples, processing circuitry of patient monitoring system 2 is configured to adjust, based at least on the analgesic agent and/or the site on patient 6 where the analgesic is administered, the nociception parameter over time. In some examples, patient monitoring system 2 may be configured to receive input indicative of the analgesic agent administered to patient 6 and/or the site on patient 6 where the analgesic is administered. In some examples, patient monitoring system 2 may communicate with an electronic medical records system to receive information indicative of the analgesic agent administered to patient 6 and/or the site on patient 6 where the analgesic is administered. In some examples, patient monitoring system 2 may store a lookup table that contains information regarding analgesia, administration sites, and how long it takes for analgesia to wear off given the different types of analgesia and different administration sites.

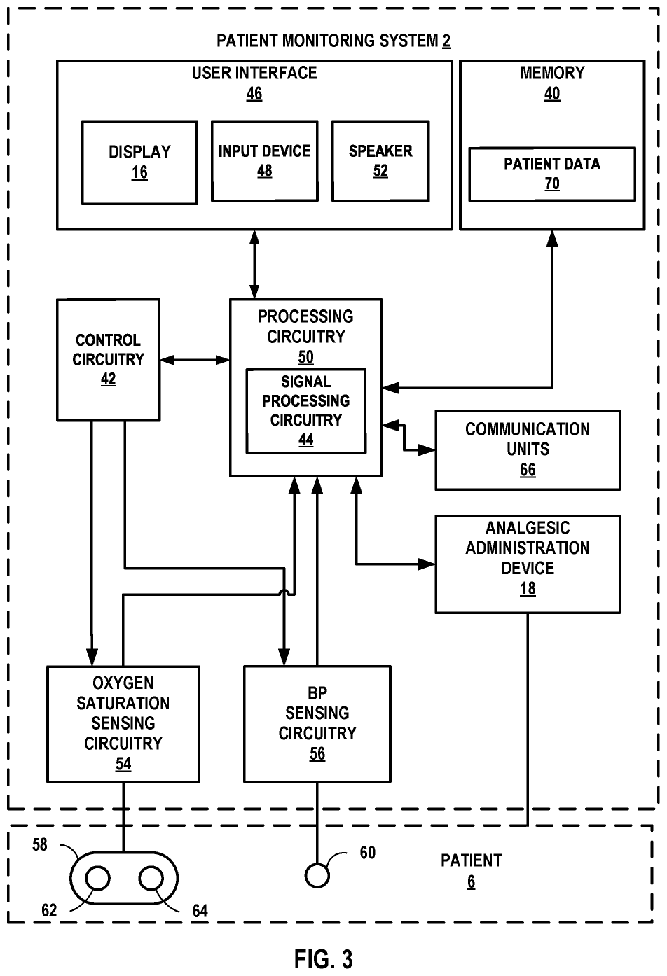

FIG. 3 is a block diagram illustrating an example of the patient monitoring system 2 of FIG. 1. As shown in FIG. 3, in some examples, patient monitoring system 2 includes memory 40, control circuitry 42, user interface 46, processing circuitry 50, sensing circuitry 54 and 56, sensing devices 58 and 60, and one or more communication units 66. In the example shown in FIG. 1, user interface 46 may include display 16, input device 48, and/or speaker 52, which may be any suitable audio device including circuitry configured to generate and output a sound and/or noise. In some examples, patient monitoring system 2 may be configured to determine and output (e.g., for display at display 16) the nociception parameter of a patient 6 during a medical procedure.

Processing circuitry 50, as well as other processors, processing circuitry, controllers, control circuitry, and the like, described herein, may include one or more processors. Processing circuitry 50 and control circuitry 42 may each include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 50 and/or control circuitry 42 may include multiple components, such as any combination of one or more microprocessors, one or more DSPs, one or more ASICs, or one or more FPGAs, as well as other discrete or integrated logic circuitry, and/or analog circuitry.

Control circuitry 42 may be operatively coupled to processing circuitry 50. Control circuitry 42 is configured to control an operation of sensing devices 58 and 60. In some examples, control circuitry 42 may be configured to provide timing control signals to coordinate operation of sensing devices 58 and 60. For example, sensing circuitry 54 and 56 may receive from control circuitry 42 one or more timing control signals, which may be used by sensing circuitry 54 and 56 to turn on and off respective sensing devices 58 and 60, such as to periodically collect calibration data using sensing devices 58 and 60. In some examples, processing circuitry 50 may use the timing control signals to operate synchronously with sensing circuitry 54 and 56. For example, processing circuitry 50 may synchronize the operation of an analog-to-digital converter and a demultiplexer with sensing circuitry 54 and 56 based on the timing control signals.

One or more communication units 66 may be operable to communicate with endotracheal tube 8 via one or more networks by transmitting and/or receiving network signals on the one or more networks such as the Internet, a Wide Area Network, a Local Area Network, and the like. Examples of one or more communication units 66 include a network interface card (e.g. such as an Ethernet card), an optical transceiver, a radio frequency transceiver, or any other type of device that can send and/or receive information. Other examples of one or more communication units 66 may include Near-Field Communications (NFC) units, Bluetooth® radios, short wave radios, cellular data radios, wireless network (e.g., Wi-Fi®) radios, as well as universal serial bus (USB) controllers.

Memory 40 may be configured to store, for example, patient data 70. For example, processing circuitry 50 may store various data associated with patient 6 in patient data 70. For example, processing circuitry 50 may store the nociception parameter of patient 6, a predetermined nociception threshold, the total amount of analgesic administered to patient 6, a current level of analgesic being administered to patient 6, and the like in patient data 70 in memory 40. The predetermined nociception threshold can be specific to patient 6 or used for a population of patients.

In some examples, memory 40 may store program instructions. The program instructions may include one or more program modules that are executable by processing circuitry 50. When executed by processing circuitry 50, such program instructions may cause processing circuitry 50 to provide the functionality ascribed to it herein. The program instructions may be embodied in software, firmware, and/or RAMware. Memory 40 may include any one or more of volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

User interface 46 may include a display 16, an input device 48, and a speaker 52. In some examples, user interface 46 may include fewer or additional components. User interface 46 is configured to present information to a user (e.g., a clinician). For example, user interface 46 and/or display 16 may include a monitor, cathode ray tube display, a flat panel display such as a liquid crystal (LCD) display, a plasma display, a light emitting diode (LED) display, and/or any other suitable display. In some examples, user interface 46 may be part of a multiparameter monitor (MPM) or other physiological signal monitor used in a clinical or other setting, a personal digital assistant, mobile phone, tablet computer, laptop computer, any other suitable computing device, or any combination thereof, with a built-in display or a separate display.

In some examples, processing circuitry 50 may be configured to present, by user interface 46, such as display 16, a graphical user interface to a user. The graphical user interface can include information regarding the delivery of analgesic or anesthesia to patient 6, one or more sensed nociception parameters, and the like. For example, the graphical user interface may include time graph 30 of FIGS. 2A-2B of the nociception parameter of patient 6 over time. In some examples, the graphical user interface can also include an instruction or suggestion to a clinician to administer additional analgesics or anesthesia or otherwise adjust the delivery of analgesics, anesthesia, or other pharmaceutical agents or fluids. User interface 46 may also include means for projecting audio to a user, such as speaker 52.

In some examples, processing circuitry 50 may also receive input signals from additional sources (not shown), such as a user. For example, processing circuitry 50 may receive from input device 48, such as a keyboard, a mouse, a touch screen, buttons, switches, a microphone, a joystick, a touch pad, or any other suitable input device or combination of input devices, an input signal. The input signal may contain information about patient 6, such as physiological parameters, treatments provided to patient 6, or the like. Additional input signals may be used by processing circuitry 50 in any of the determinations or operations it performs in accordance with examples described herein. For example, the input processing circuitry 50 receives via input device 48 can indicate the occurrence of a medical event, based on which processing circuitry 50 may determine a patient-specific nociception threshold.

In some examples, processing circuitry 50 and user interface 46 may be part of the same device or supported within one housing (e.g., a computer or monitor). In other examples, processing circuitry 50 and user interface 46 may be separate devices configured to communicate through a wired connection or a wireless connection.

Sensing circuitry 54 and 56 is configured to receive signals (“physiological signals”) indicative of physiological parameters from respective sensing devices 58 and 60 and communicate the physiological signals to processing circuitry 50. Sensing devices 58 and 60 may include any sensing hardware configured to sense a physiological parameter of a patient, e.g., indicative of a nociception response of patient 6. Example sensing hardware includes, but is not limited to, one or more electrodes, light sources, optical receivers, blood pressure cuffs, or the like. The sensed physiological signals may include signals indicative of physiological parameters from a patient, such as, but not limited to, blood pressure, blood oxygen saturation (e.g., pulse oximetry and/or regional oxygen saturation), blood volume, heart rate, heart rate variability, skin conductance, and respiration. For example, sensing circuitry 54 and 56 may include, but are not limited to, blood pressure sensing circuitry, blood oxygen saturation sensing circuitry, blood volume sensing circuitry, heart rate sensing circuitry, temperature sensing circuitry, electrocardiography (ECG) sensing circuitry, electroencephalogram (EEG) sensing circuitry, electromyogram (EMG) sensing circuitry or any combination thereof.

In some examples, sensing circuitry 54 and 56 and/or processing circuitry 50 may include signal processing circuitry 44 configured to perform any suitable analog conditioning of the sensed physiological signals. For example, sensing circuitry 54 and 56 may communicate to processing circuitry 50 an unaltered (e.g., raw) signal. Processing circuitry 50, e.g., signal processing circuitry 44, may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

In some examples, the conditioned analog signals may be processed by an analog-to-digital converter of signal processing circuitry 44 to convert the conditioned analog signals into digital signals. In some examples, signal processing circuitry 44 may operate on the analog or digital form of the signals to separate out different components of the signals. In some examples, signal processing circuitry 44 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. In some examples, signal processing circuitry 44 may decrease the number of samples in the digital detector signals. In some examples, signal processing circuitry 44 may remove dark or ambient contributions to the received signal. Additionally, or alternatively, sensing circuitry 54 and 56 may include signal processing circuitry 44 to modify one or more raw signals and communicate to processing circuitry 50 one or more modified signals.

In the example shown in FIG. 3, patient monitoring system 2 includes an oxygen saturation sensing device 58 (also referred to herein as blood oxygen saturation sensing device 58), which is configured to generate an oxygen saturation signal indicative of blood oxygen saturation within the venous, arterial, and/or capillary systems within a region of patient 6. For example, oxygen saturation sensing device 58 may include a sensor configured to non-invasively generate a plethysmography (PPG) signal. One example of such a sensor may be one or more oximetry sensors (e.g., one or more pulse oximetry sensors) placed at one or multiple locations on patient 6, such as at a fingertip of patient 6, an earlobe of patient 6, and the like.

In some examples, oxygen saturation sensing device 58 may be configured to be placed on the skin of patient 6 to determine regional oxygen saturation of a particular tissue region, e.g., the frontal cortex or another cerebral location of patient 6. Oxygen saturation sensing device 58 may include emitter 62 and detector 64. Emitter 62 may include at least two light emitting diodes (LEDs), each configured to emit at different wavelengths of light, e.g., red or near infrared light. As used herein, the term “light” may refer to energy produced by radiative sources and may include any wavelength within one or more of the ultrasound, radio, microwave, millimeter wave, infrared, visible, ultraviolet, gamma ray or X-ray electromagnetic radiation spectra. In some examples, light drive circuitry (e.g., within sensing device 58, sensing circuitry 54, control circuitry 42, and/or processing circuitry 50) may provide a light drive signal to drive emitter 62 and to cause emitter 62 to emit light. In some examples, the LEDs of emitter 62 emit light in the range of about 600 nanometers (nm) to about 1000 nm. In a particular example, one LED of emitter 62 is configured to emit light at about 730 nm and the other LED of emitter 62 is configured to emit light at about 810 nm. Other wavelengths of light may be used in other examples.

Detector 64 may include a first detection element positioned relatively “close” (e.g., proximal) to emitter 62 and a second detection element positioned relatively “far” (e.g., distal) from emitter 62. In some examples, the first detection elements and the second detection elements may be chosen to be specifically sensitive to the chosen targeted energy spectrum of emitter 62. Light intensity of multiple wavelengths may be received at both the “close” and the “far” detector 64. For example, if two wavelengths are used, the two wavelengths may be contrasted at each location and the resulting signals may be contrasted to arrive at an oxygen saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). In operation, light may enter detector 64 after passing through the tissue of patient 6, including skin, bone, other shallow tissue (e.g., non-cerebral tissue and shallow cerebral tissue), and/or deep tissue (e.g., deep cerebral tissue). Detector 64 may convert the intensity of the received light into an electrical signal. The light intensity may be directly related to the absorbance and/or reflectance of light in the tissue. Surface data from the skin and skull may be subtracted out, to generate an oxygen saturation signal for the target tissues over time.

Oxygen saturation sensing device 58 may provide the oxygen saturation signal to processing circuitry 50. Additional example details of determining oxygen saturation based on light signals may be found in commonly assigned U.S. Pat. No. 9,861,317, which issued on Jan. 9, 2018, and is entitled “Methods and Systems for Determining Regional Blood Oxygen Saturation.” One example of such an oxygen saturation signal may be a plethysmography (PPG) signal.

In the example shown in FIG. 3, patient monitoring system 2 includes a blood pressure sensing device 60, which is configured to generate a blood pressure signal indicative of a blood pressure of patient 6. For example, blood pressure sensing device 60 may include a blood pressure cuff configured to non-invasively sense blood pressure or an arterial line configured to invasively monitoring blood pressure in an artery of patient 6. In some examples, the blood pressure signal may include at least a portion of a waveform of the acquisition blood pressure. Blood pressure sensing device 60 may be configured to generate a blood pressure signal indicative of the blood pressure of patient over time. Blood pressure sensing device 60 may provide the blood pressure signal to sensing circuitry 56, processing circuitry 50, or to any other suitable processing device, which may be part of patient monitoring system 2 or a device separate from patient monitoring system 2, such as another device co-located with patient monitoring system 2 or remotely located relative to patient monitoring system 2.

In operation, blood pressure sensing device 60 and oxygen saturation sensing device 58 may each be placed on the same or different parts of the body of patient 6. For example, blood pressure sensing device 60 and oxygen saturation sensing device 58 may be physically separate from each other and may be separately placed on patient 6. As another example, blood pressure sensing device 60 and oxygen saturation sensing device 58 may in some cases be supported by a single sensor housing. One or both of blood pressure sensing device 60 or oxygen saturation sensing device 58 may be further configured to measure other patient parameters, such as hemoglobin, respiratory rate, respiratory effort, heart rate, saturation pattern detection, response to stimulus such as bispectral index (BIS) or electromyography (EMG) response to electrical stimulus, or the like. While an example patient monitoring system 2 is shown in FIG. 3, the components illustrated in FIG. 3 are not intended to be limiting. Additional or alternative components and/or implementations may be used in other examples.

Processing circuitry 50 may be configured to receive one or more physiological signals generated by sensing devices 58 and 60 and sensing circuitry 54 and 56. The physiological signals may include a signal indicating blood pressure and/or a signal, such as a PPG signal, indicating oxygen saturation. Processing circuitry 50 may be configured to obtain the nociception parameter for patient 6 over time while patient 6 is in surgery by continuously determining, based on the one or more physiological signals generated by sensing devices 58 and 60, the nociception parameter for patient 6. For example, the nociception parameter may be a value between 0 to 100 that indicates the amount of surgical stress experienced by patient 6 during surgery. As processing circuitry 50 receives the one or more physiological signals during surgery of patient 6, processing circuitry 50 may be able to periodically or continuously determine, based on the one or more physiological signals, the nociception parameter for patient 6 over time. As such processing circuitry 50, sensing circuitry 54 and 56, and sensing devices 58 and 60 may together implement nociception monitor 4 of patient monitoring system 2 shown in FIG. 1. In other examples, processing circuitry 50 may be configured to obtain the nociception parameter for patient 6 via one or more external devices. For example, processing circuitry 50 may be configured to communicate, via communication units 66, with an external device that sends the nociception parameter for patient 6 to processing circuitry 50.

In accordance with aspects of the present disclosure, processing circuitry 50 is configured to adaptively determine, based on the nociception parameter of patient 6 that corresponds to an intubation period, a patient-specific nociception threshold for patient 6 that is adapted for a nociceptive response of patient 6 to stimuli. Further, processing circuitry 50 is also configured to determine, based at least in part on comparing the nociception parameter of patient 6 with the determined nociception threshold, whether to adjust the amount of analgesic administered to patient 6.

Processing circuitry 50 is configured to receive an indication of a medical event using any suitable technique. In some examples, processing circuitry 50 may be configured to receive, via one or more communication units 66 and from endotracheal tube 8, an indication of patient 6 being intubated with endotracheal tube 8 (i.e., an intubation event). For example, processing circuitry 50 may receive an indication of sensor values from endotracheal tube 8 and may determine, based at least in part on the sensor values, determine that an intubation event has occurred.

Processing circuitry 50 may be configured to determine a set of nociception parameter of patient 6 that corresponds to the intubation event. That is, processing circuitry 50 may be configured to determine the nociceptive response of patient 6 to the intubation event. Processing circuitry 50 may be configured to determine, based at least in part on a set of nociception parameters of patient 6 that corresponds to the intubation event, a nociception threshold for the patient. Specifically, processing circuitry 50 may be configured to derive a characteristic nociception parameter (NPS) for patient 6 based at least in part on the set of nociception parameters of patient 6 during the intubation period, and may be configured to determine a nociception threshold for patient 6 based at least in part on the characteristic nociception parameter for patient 6.

In some examples, processing circuitry 50 may be configured to determine a characteristic nociception parameter for patient 6 as a multiple of the mathematical mean of the set nociception parameters that corresponds to the intubation event (or other medical even). In some examples, processing circuitry 50 may be configured to determine a characteristic nociception parameter for patient 6 as a percentile of the set of nociception parameters that corresponds to the intubation event. Processing circuitry 50 may also be configured to use any other suitable distance metrics and multiples thereof to derive a characteristic nociception parameter for patient 6 based at least in part on the set of nociception parameters of patient 6 that corresponds to the intubation event.

In some examples, to determine a nociception threshold for patient 6 based at least in part on the characteristic nociception parameter for patient 6, processing circuitry 50 may be configured to determine the nociception threshold for patient 6 as the characteristic nociception parameter for patient 6. That is, processing circuitry 50 may be configured to set the value of the nociception threshold for patient 6 to the value of the characteristic nociception parameter for patient 6. In other examples, processing circuitry 50 may be configured to determine the nociception threshold for patient 6 as a percentage of the characteristic nociception parameter for patient 6 or as a multiple of the characteristic nociception parameter for patient 6.

Processing circuitry 50 may also be configured to adjust the nociception threshold based on various factors, as discussed above. For example, if one or more sensors 10 coupled to endotracheal tube 8 comprises force sensors that measure the amount of force exerted on patient 6 by intubating and/or extubating endotracheal tube 8, processing circuitry 50 may be configured to adjust the nociception threshold based on the amount of force exerted on patient 6 by intubating and/or extubating endotracheal tube 8 as measured by the force sensors. If, for example, processing circuitry 50 determines that the force sensors measured a relatively low amount of force, e.g., a force less than or equal to a force threshold stored by memory 40 or a memory of another device, during intubation of patient 6 with endotracheal tube 8, then processing circuitry 50 may be configured to adjust the nociception threshold by raising the nociception threshold to account for the relatively lower nociception response of patient 6 due to intubating endotracheal tube 8. In some examples, processing circuitry 50 raises the nociception threshold by a first predetermined amount, which can also be stored by memory 40 or a memory of another device. In contrast, if processing circuitry 50 determines that the force sensors measured a relatively high amount of force during intubation of patient 6 with endotracheal tube 8, e.g., a force greater than the force threshold, then processing circuitry of 50 lower the nociception threshold to account for the relatively higher nociception response of patient 6 due to intubating endotracheal tube 8. In some examples, processing circuitry 50 lowers the nociception threshold by a second predetermined amount, which can also be stored by memory 40 or a memory of another device. The first and second predetermined amounts can be equal in some examples and different in other examples.

In some examples, processing circuitry 50 may be configured to adjust the nociception threshold based on the amount of analgesic already administered to patient 6 prior to the intubation period for patient 6. For example, a patient having a relatively higher analgesic load may not exhibit as large of a nociception response to intubation as a patient having a relatively lower analgesic load. As such, if processing circuitry 50 determines that the amount of analgesic administered to patient 6 prior to the intubation period is relatively high, e.g., greater than or equal to a predetermined analgesic threshold stored by memory 40 or a memory of another device, then processing circuitry 50 may be configured to adjust the nociception threshold by raising the nociception threshold. In some examples, processing circuitry 50 raises the nociception threshold by a first predetermined amount, which can also be stored by memory 40 or a memory of another device.

Conversely, if processing circuitry 50 determines that the amount of analgesic administered to patient 6 prior to the intubation period is relatively low, e.g., less than the predetermined analgesic threshold, then processing circuitry 50 may be configured to adjust the nociception threshold by lowering the nociception threshold. In some examples, processing circuitry 50 lowers the nociception threshold by a second predetermined amount, which can also be stored by memory 40 or a memory of another device. The first and second predetermined amounts can be equal in some examples and different in other examples.

In some examples, processing circuitry 50 is configured to adjust the nociception threshold over time. For example, as the analgesic previously administered to patient 6 wears off over time, processing circuitry 50 may be configured to adjust the nociception threshold over time. As an example, processing circuitry 50 may be configured to lower the nociception threshold as the analgesic is expected to wear off over time, as the stress response of patient 6 to a particular stimulus may increase as the analgesic wears off. In some examples, processing circuitry 50 may lower the nociception parameter threshold according to a predetermined rate of change, which specifies a correspondence between an amount by which the lower the nociception threshold and time. The predetermined rate of change can be stored by memory 40 or a memory of another device.

Processing circuitry 50 may be configured to adjust, based at least on the analgesic agent and/or the site on patient 6 where the analgesic is administered, the nociception parameter over time. In some examples, processing circuitry 50 may be configured to receive input indicative of the analgesic agent administered to patient 6 and/or the site on patient 6 where the analgesic is administered and to store such information, such as in memory 40. In some examples, processing circuitry 50 may be configured to communicate with an electronic medical records system to receive information indicative of the analgesic agent administered to patient 6 and/or the site on patient 6 where the analgesic is administered and to store such information, such as in memory 40.

In some examples, processing circuitry of 50 may be configured to determine the characteristic nociception parameter for patient 6 at multiple points during a medical procedure and to determine the nociception threshold for patient 6 based at least in part on the determined characteristic nociception parameter at those points during the medical procedure. For example, at a point in time during the medical procedure, processing circuitry 50 may be configured to determine the nociception parameter during a period of time that immediately precedes the point in time, and may be configured to determine the characteristic nociception parameter based on the nociception parameter during that period of time. Processing circuitry 50 may then determine the nociception threshold for patient 6 based at least in part on the determined characteristic nociception parameter.

As processing circuitry 50 monitors the nociception parameter of patient 6, processing circuitry 50 may compare the nociception parameter of patient 6 to the nociception threshold (stored by memory 40 or a memory of another device) for patient 6 to detect a nociception event. For example, processing circuitry 50 may determine whether the nociception parameter of patient 6 is greater than or equal to the nociception threshold for patient 6. Processing circuitry 50 may, in response to determining that the nociception parameter of patient 6 is greater than or equal to a nociception threshold for patient 6, determine that a nociception event has occurred.

In some examples, processing circuitry 50 may, in response to determining that the nociception event has occurred, output a notification via user interface 46. The notification can be any suitable visual, audible, somatosensory, or any combination thereof, notification that indicates the nociception event was detected. In some examples, the notification includes an indication to adjust an amount of analgesic to administer to patient 6. That is, processing circuitry 50 may cause analgesic administration device 18 to increase the amount of analgesic administered to patient 6 to dampen the surgical stress experienced by patient 6 by directly controlling analgesic administration device 18 or by generating a notification that causes a clinician to control analgesic administration device 18. Example analgesics that analgesic administration device 18 can administer include, but are not limited to, one or more of remifentanil, alfentanil, and fentanyl.

In some examples, to provide an indication to adjust an amount of analgesic to administer to patient 6, processing circuitry 50 may output, for display at display 16, an indication to increase an amount of analgesic to administer to patient 6, so that a clinician that views display 16 may therefore control analgesic administration device 18 to adjust the amount of analgesic administered to patient 6.

In some examples, to provide an indication to adjust an amount of analgesic to administer to patient 6, processing circuitry 50 may send, to analgesic administration device 18, the indication to adjust the amount of analgesic administered to patient 6. Analgesic administration device 18 may, in response to receiving the indication, adjust the amount of analgesic that analgesic administration device 18 delivers to patient 6. In this way, patient monitoring system 2 may act as an automated analgesic administration system.

In some examples, processing circuitry 50 may determine how much to adjust the amount of analgesic administered to patient 6 based on at least one of: a current amount of analgesic being administered to patient 6 and a total amount of analgesic administered to patient 6 during surgery. In some examples, it may be desirable to control the amount of analgesic being administered to patient 6 so that the amount at any point in time does not exceed a specified analgesic level. Thus, processing circuitry 50 may determine whether increasing the current amount of analgesic administered to patient 6 may cause the amount of analgesic administered to exceed the specified analgesic level and, if so, to reduce the increase in the amount of analgesic administered to patient 6 so that the amount of analgesic administered to patient 6 remains below the specified analgesic level.

In some examples, it may be desirable to limit to the total amount of analgesic administered to patient 6 during surgery. Thus, in some examples, processing circuitry 50 may determine whether increasing the current amount of analgesic administered to patient 6 may cause the total amount of analgesic administered to patient 6 during surgery to exceed the limit and, if so, to reduce the increase in the amount of analgesic administered to patient 6 so that the amount of analgesic administered to patient 6 does not cause the total amount of analgesic administered to patient 6 during surgery to exceed the limit.

The components of patient monitoring system 2 that are shown and described as separate components are shown and described as such for illustrative purposes only. In some examples the functionality of some of the components may be combined in a single component. For example, the functionality of processing circuitry 50 and control circuitry 42 may be combined in a single processor system. Additionally, in some examples the functionality of some of the components of patient monitoring system 2 shown and described herein may be divided over multiple components or over multiple devices. For example, some or all of the functionality of control circuitry 42 may be performed in processing circuitry 50, or sensing circuitry 54 and 56. In other examples, the functionality of one or more of the components may be performed in a different order or may not be required.

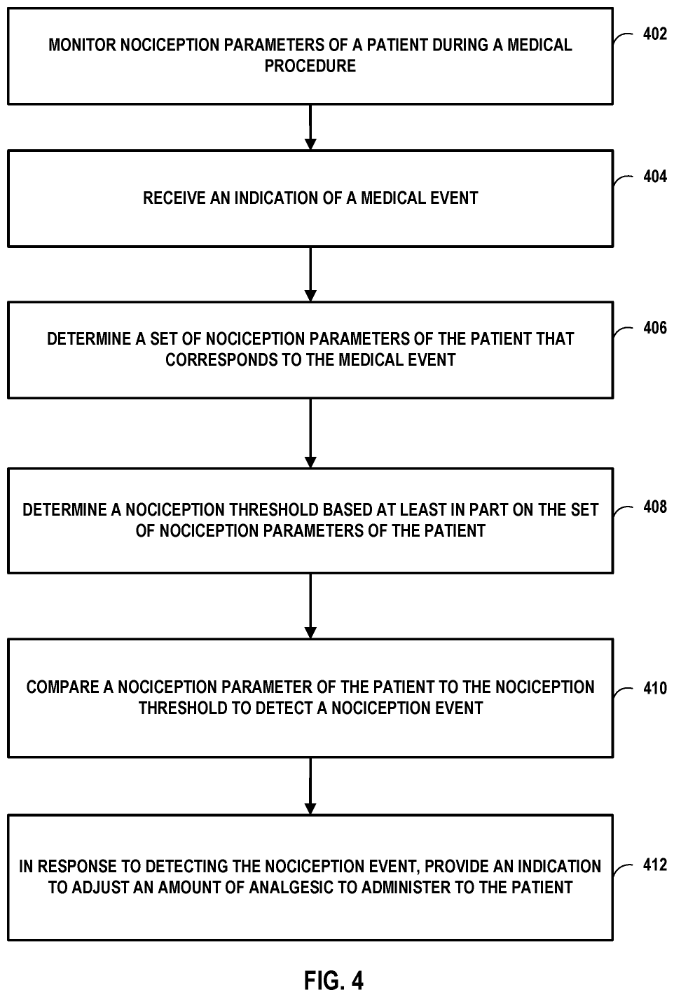

FIG. 4 is a flow diagram illustrating an example method of determining a patient-specific nociception threshold. Although FIG. 4 is described with respect to processing circuitry 50 of patient monitoring system 2 (FIGS. 1 and 3), in other examples, different processing circuitry, alone or in combination with processing circuitry 50, may perform any part of the technique of FIG. 4.

As shown in FIG. 4, processing circuitry 50 may monitor nociception parameters of a patient during a medical procedure (402). Processing circuitry 50 may receive an indication of a medical event (404). For example, the medical event may be an intubation event or an extubation event during the medical procedure and processing circuitry 50 may receive the indication of the medical event from endotracheal tube 8 or from a user via input device 48 or a different user input device , such as a user input mechanism (e.g., a button or switch) on endotracheal tube . As another example, the medical event may be an incision event and processing circuitry 50 may receive the indication of the medical event from a user via input device 48 or a different user input device, or from a surgical robot. As another example, the medical event may be delivery of electrical stimulation to patient 6 and processing circuitry 50 may receive the indication of the medical event from the electrical stimulation device or from a user via input device 48 a different user input device. Other types of medical events may apply in other examples.

Processing circuitry 50 may determine a set of nociception parameters of the patient 6 that corresponds to the medical event (406). Processing circuitry 50 may determine a nociception threshold based at least in part on the set of nociception parameters of the patient 6 (408). Processing circuitry 50 may compare a nociception parameter of the patient 6 to the nociception threshold to detect a nociception event (410). Processing circuitry 50 may, in response to determining the nociception event, provide an indication via display 16, an audio device including audio generating circuitry, a somatosensory device, or another output device, to adjust an amount of analgesic administered to the patient 6 (412).

The disclosure includes the following examples.

Example 1: A method includes monitoring, by processing circuitry, nociception parameters of a patient during a medical procedure; receiving, by the processing circuitry, an indication of a medical event; determining, by the processing, circuitry, a set of nociception parameters of the patient that corresponds to the medical event; determining, by the processing circuitry, a nociception threshold based at least in part on the set of nociception parameters of the patient; comparing, by the processing circuitry, a nociception parameter of the patient to the nociception threshold to detect a nociception event; and in response to detecting the nociception event, providing, by the processing circuitry, an indication to adjust an amount of analgesic administered to the patient.

Example 2: The method of example 1, wherein determining the nociception threshold further comprises: determining, by the processing circuitry, a characteristic nociception parameter of the patient based at least in part on the set of nociception parameters of the patient that corresponds to the medical event; and determining, by the processing circuitry, the nociception threshold based at least in part on the characteristic nociception parameter of the patient.

Example 3: The method of example 2, wherein determining the characteristic nociception parameter of the patient based at least in part on the set of nociception parameters of the patient comprises: determining, by the processing circuitry, the characteristic nociception parameter as a specified percentile of the set of nociception parameters.
Example 4: The method of example 2, wherein determining the characteristic nociception parameter of the patient based at least in part on the set of nociception parameters of the patient comprises: determining, by the processing circuitry, the characteristic nociception parameter as a multiple of a mean nociception parameter of the set of nociception parameters.

Example 5: The method of any of examples 1-4, wherein receiving the indication of the medical event comprises: receiving, by the processing circuitry from an endotracheal tube, an indication of one or more sensor values; and determining, by the processing circuitry, an occurrence of an intubation event based at least in part on the one or more sensor values.

Example 6: The method of example 5, wherein determining the occurrence of the intubation event based at least in part on the one or more sensor values comprises: determining, by the processing circuitry, a rate of change in the one or more sensor values that is above a rate threshold.

Example 7: The method of any of examples 5 and 6, wherein the one or more sensor values indicate an amount of force associated with intubating the patient.

Example 8: The method of example 7, further includes adjusting, by the processing circuitry, the nociception threshold based at least in part on the amount of force associated with intubating the patient.

Example 9: The method of example 8, wherein adjusting the nociception threshold based at least in part on the amount of force associated with intubating the patient comprises: in response to determining that the amount of force associated with intubating the patient is less than or equal to a predetermined force threshold, raising, by the processing circuitry, the nociception threshold.

Example 10: The method of example 8, wherein adjusting the nociception threshold based at least in part on the amount of force associated with intubating the patient further comprises: in response to determining that the amount of force associated with intubating the patient is relatively high, lowering, by the processing circuitry, the nociception threshold.

Example 11: The method of any of examples 1-10, further includes adjusting, by the processing circuitry, the nociception threshold based at least in part on an analgesic load of the patient.

Example 12: The method of example 11, wherein adjusting the nociception threshold based at least in part on the analgesic load of the patient further comprises: in response to determining that the analgesic load of the patient is above a high analgesic load threshold, raising, by the processing circuitry, the nociception parameter threshold.

Example 13: The method of any of examples 1-12, further includes adjusting, by the processing circuitry, the nociception threshold over time.

Example 14: The method of example 13, wherein adjusting the nociception threshold based at least in part on the analgesic load of the patient further comprises: in response to determining that the analgesic load of the patient is at or below a low analgesic load threshold, lowering, by the processing circuitry, the nociception threshold.

Example 15: The method of any of examples 1-14, wherein receiving the indication of the medical event comprises receiving an indication of the patient being incised.

Example 16: The method of any of examples 1-14, wherein receiving the indication of the medical event comprises receiving an indication of a delivery of tetanic stimulation to the patient.

Example 17: A system includes memory; and processing circuitry configured to perform any combination of the method of claims 1-16.

Example 18: A non-transitory computer readable storage medium comprising instructions that, when executed, cause processing circuitry to perform any combination of the method of examples 1-16.

The techniques described in this disclosure, including those attributed to patient monitoring system 2, processing circuitry 50, control circuitry 42, sensing circuitries 54, 56, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example.

In one or more examples, the functions described in this disclosure may be implemented in hardware, software, firmware, or any combination thereof If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

1. A system comprising:
memory; and
processing circuitry operably coupled to the memory and configured to:
monitor nociception parameters of a patient during a medical procedure;
receive an indication of a medical event;
determine a set of nociception parameters of the patient that corresponds to the medical event;
determine a nociception threshold based at least in part on the set of nociception parameters of the patient;
compare a nociception parameter of the patient to the nociception threshold to detect a nociception event; and
in response to detecting the nociception event, provide an indication to adjust an amount of analgesic administered to the patient.

2. The system of claim 1, wherein to determine the nociception threshold, the processing circuitry is further configured to:
determine a characteristic nociception parameter of the patient based at least in part on the set of nociception parameters of the patient that corresponds to the medical event; and
determine the nociception threshold based at least in part on the characteristic nociception parameter of the patient.

3. The system of claim 2, wherein to determine the characteristic nociception parameter of the patient based at least in part on the set of nociception parameters of the patient, the processing circuitry is further configured to:
determine the characteristic nociception parameter as a specified percentile of the set of nociception parameters.

4. The system of claim 2, wherein to determine the characteristic nociception parameter of the patient based at least in part on the set of nociception parameters of the patient, the processing circuitry is further configured to:
determine the characteristic nociception parameter as a multiple of a mean nociception parameter of the set of nociception parameters.

5. The system of claim 1, wherein to receive the indication of the medical event, the processing circuitry is further configured to:
receive, from an endotracheal tube, an indication of one or more sensor values; and
determine an occurrence of an intubation event based at least in part on the one or more sensor values.

6. The system of claim 5, wherein to determine the occurrence of the intubation event based at least in part on the one or more sensor values, the processing circuitry is further configured to:
determine a rate of change in the one or more sensor values that is above a rate threshold.

7. The system of claim 5, wherein the one or more sensor values indicate an amount of force associated with intubating the patient.

8. The system of claim 7, wherein the processing circuitry is further configured to:
adjust the nociception threshold based at least in part on the amount of force associated with intubating the patient.

9. The system of claim 8, wherein to adjust the nociception threshold based at least in part on the amount of force associated with intubating the patient, the processing circuitry is further configured to:
in response to determining that the amount of force associated with intubating the patient is less than or equal to a predetermined force threshold, raise the nociception threshold.

10. The system of claim 8, wherein to adjust the nociception threshold based at least in part on the amount of force associated with intubating the patient, the processing circuitry is further configured to:
in response to determining that the amount of force associated with intubating the patient is relatively high, lower the nociception threshold.

11. The system of claim 1, wherein the processing circuitry is further configured to adjust the nociception threshold based at least in part on an analgesic load of the patient.

12. The system of claim 11, wherein to adjust the nociception threshold based at least in part on the analgesic load of the patient, the processing circuitry is further configured to:
in response to determining that the analgesic load of the patient is above a high analgesic load threshold, raise the nociception threshold.

13. The system of claim 11, wherein to adjust the nociception threshold based at least in part on the analgesic load of the patient, the processing circuitry is further configured to:
in response to determining that the analgesic load of the patient is at or below a low analgesic load threshold, lower the nociception threshold.

14. The system of claim 1, wherein the processing circuitry is further configured to adjust the nociception threshold over time.

15. The system of claim 1, wherein the indication of the medical event comprises an indication of the patient being incised or an indication of a delivery of tetanic stimulation to the patient.

16. A method comprising:
monitoring, by processing circuitry, nociception parameters of a patient during a medical procedure;
receiving, by the processing circuitry, an indication of a medical event;
determining, by the processing circuitry, a set of nociception parameters of the patient that corresponds to the medical event;
determining, by the processing circuitry, a nociception threshold based at least in part on the set of nociception parameters of the patient;
comparing, by the processing circuitry, a nociception parameter of the patient to the nociception threshold to detect a nociception event; and
in response to detecting the nociception event, providing, by the processing circuitry, an indication to adjust an amount of analgesic administered to the patient.

17. The method of claim 16, wherein determining the nociception threshold further comprises:
determining, by the processing circuitry, a characteristic nociception parameter of the patient based at least in part on the set of nociception parameters of the patient that corresponds to the medical event; and
determining, by the processing circuitry, the nociception threshold based at least in part on the characteristic nociception parameter of the patient.

18. The method of claim 17, wherein determining the characteristic nociception parameter of the patient based at least in part on the set of nociception parameters of the patient further comprises:
determining, by the processing circuitry, the characteristic nociception parameter as a specified percentile of the set of nociception parameters.

19. The method of claim 17, wherein determining the characteristic nociception parameter of the patient based at least in part on the set of nociception parameters of the patient further comprises:
determining, by the processing circuitry, the characteristic nociception parameter as a multiple of a mean nociception parameter of the set of nociception parameters.

20. A non-transitory computer readable storage medium comprising instructions that, when executed, cause processing circuitry to:
monitor nociception parameters of a patient during a medical procedure;
receive an indication of a medical event;
determine a set of nociception parameters of the patient that corresponds to the medical event;
determine a nociception threshold based at least in part on the set of nociception parameters of the patient;
compare a nociception parameter of the patient to the nociception threshold to detect a nociception event; and
in response to detecting the nociception event, provide an indication to adjust an amount of analgesic administered to the patient.
